(19) United States
(12) Patent Application Publication
(10) Pub. No.: US 2026/0130800 A1
(43) Pub. Date: May 14, 2026

(54) Compression Legwear with Linear Pads to Control Symptoms of Varicose Veins

(71) Applicant: Norman Noguera, Sherman Oaks, CA (US)

(72) Inventor: Norman Noguera, Sherman Oaks, CA (US)

(21) Appl. No.: 19/086,009

(22) Filed: Mar. 20, 2025

Related U.S. Application Data
(63) Continuation-in-part of application No. 18/941,607, filed on Nov. 8, 2024, now Pat. No. 12,521,281.

(51) Int. Cl. A61F 13/08

(52) U.S. Cl. CPC A61F 13/085

(57) ABSTRACT

Knee-high and thigh-high compression legwear for right and left legs are disclosed. The legwear, made to be worn by people who have varicose veins, includes at least one linear pad sized, adapted, and configured to exert a pressure of 20-30 mm Hg along a varicose vein, and a zipper for relieving pressure, enabling a wearer to easily remove the legwear.

### REFERENCE TO RELATED APPLICATION

This nonprovisional patent application is a continuation-in-part of nonprovisional patent application bearing U.S. Ser. No. 18/941,607 (filed Nov. 8, 2024), hereby incorporated by reference for purposes of priority pursuant to 35 US Code, § 120.

### FIELD

The present subject matter, generally directed to a compression garment, is more particularly directed to compression legwear with pads to control varicose vein symptoms.

### BACKGROUND

Compression garments, articles of clothing that fit tightly around the skin of the wearer and are available in varying degrees of compression, include legwear providing a compression of 20-30 mm Hg or higher, which typically require a prescription by a doctor.

Compression garments and such are known. U.S. Pat. No. 5,179,941 to Siemssen et al. discloses contractile sleeves for peristaltic extremity treatment. U.S. Pat. No. 6,080,120 to Sandman et al. discloses a compression sleeve for gradient sequential compression systems. U.S. Pat. No. 6,254,554 to Turtzo is for a compression sleeve to treat lymphedema. U.S. Pat. No. 6,613,007 to Reid, Jr. discloses multilayer compression stockings. U.S. Pat. No. 6,725,691 to Yakopson discloses a knitted therapeutic medical compression stocking manufactured from courses of bi-component inlaid courses of spandex yarn. U.S. Pat. No. 7,028,690 to Schneider et al. discloses a compression stocking manufactured from a knit or woven elastic fabric provided with a compression support body. U.S. Pat. No. 7,562,541 to Hermanson et al. discloses therapeutic compression stockings knitted in an integrated knit format to have an oversized heel pocket from which an ankle arch portion extends, and which is knitted in a rib stitch format that is relatively free of wrinkles when donned. U.S. Pat. No. 10,172,743 to Wright et al. discloses a compression element comprising a core of fluid-filled cells and an outer layer of material adapted for maintaining contact with skin.

Varicose veins are swollen, twisted, blood vessels that are often seen bulging just under the surface of skin of a person. Varicose veins can be seen as blue or purple bulges, which usually appear in the legs, the feet, and the ankles. Varicose veins can be painful or itchy. When the veins in the legs of a person become enlarged, the valves in such veins often do not function properly and may cause bi-directional blood flow called “valvular reflux” in such veins. Moreover, as blood in such veins flows toward the feet, a condition called “venous hypertension” could develop, causing unpleasant symptoms including pain, swelling, skin discoloration, and in certain worst-case scenarios, “venous ulcers.”

Graduated compression hosiery has long been used in the medical industry, for example, to aid in prevention of deep vein thrombosis and help manage post-thrombotic syndrome. Also, graduated compression hosiery has known applications in managing varicose veins and, when used for everyday wear, may help in terms of preventing tired, swollen, and generally uncomfortable legs, especially when one is traveling and pregnant.

One way of controlling the varicose veins of patients thus afflicted is to manage the symptoms of the venous valvular reflux in their legs by recommending or prescribing such patients wear special medical grade (20-30 mm Hg) firm compression stockings. However, many patients state they find such stockings difficult to put on or uncomfortable to wear, due in part to circumferential pressure inwardly directed around their legs. With traditional compression stockings, an entire length of leg must be tightly compressed to decompress the varicose veins, which may explain why people do not want to wear them.

In legs, the most common veins prone to developing valvular reflux are the great and small saphenous veins, both referred to as superficial veins since they are close to the skin surface. Unfortunately, due to their location, the valves associated with superficial veins often fail since the valves are not protected by deep muscles and associated tissue as are deep veins; and another risk factor for developing varicose veins is family history.

U.S. Pat. No. 10,781,542 to Gaither discloses a knit sock including a foot portion having an upper instep area and a lower sole area integrally knit of a body yarn in axial wales and circumferential courses. The lower sole area defines inner and outer arch regions. The inner arch region includes a targeted compression zone adapted to reside generally adjacent to an inner arch of a foot of a wearer of the sock. The compression zone includes axially extending compression ridges of variable length spaced apart from a top portion of the compression zone to a bottom portion of the zone. When the sock is worn, a compression force applied by the foot portion within the targeted compression zone is greater than a compression force in adjacent areas of the foot portion of the sock.

NZ 710409A, in general, discloses anatomically targeted compression clothing, and more particularly, discloses an item of clothing, adapted to be worn against the skin. The item of clothing adapted to be worn against the skin comprises at least one panel adapted to provide targeted compression of at least 20% of the total length of a surface vein selected from the cephalic vein or the basilic vein in the arm; the short saphenous vein or the long saphenous vein in the leg; the collection of drainage plexuses in the buttocks and lateral aspect of the hips; or the drainage plexus in the anterior chest wall overlying the pectoral muscles; or a combination thereof. The panel is located on the inside of the item of clothing and comprises a singular or plurality of protrusion(s) or pads, projecting inwardly towards the skin surface such that they act on the body, in use, to thereby provide a targeted compression. U.S. Pat. No. 10,172,743 to Wright et al. discloses a compression element comprising a core of fluid-filled cells and an outer layer of material adapted for maintaining contact with skin. An embodiment comprises a folded or rolled cylindrical core of air or nitrogen-filled bubble-wrap material covered by a skin-compatible bandaging material. The compression elements described are said to provide consistent compression to a blood vessel after endovenous endothelial wall-damaging techniques.

My careful assessment of the two prior art references (summarized immediately above) is as follows: They claim compression elements/stockings can be used to treat chronic venous insufficiency, or varicose veins. After assessing them, I believe this is a false claim. There are, for instance, no known compression stockings or sleeves that can treat chronic venous sufficiency or varicose veins. Compression stockings and sleeves are used as a conservative therapy. This means such stockings and sleeves can only provide temporary relief to wearers. Chronic venous insufficiency means varicose veins disease has existed and progressed over a length of time. As noted in this specification, the cause of chronic venous insufficiency in the lower extremity is valvular reflux in the saphenous veins. Currently, there is no known technology or procedure available to fix or treat incompetent valves in veins. A defective or refluxing venous valve is an irreversible medical condition. While endovenous laser treatment is a procedure available to treat abnormal saphenous veins, even laser ablation does not treat leaky valves. Rather, laser ablation treatment is a procedure that permanently closes abnormal saphenous veins.

Therefore, to be clear, the present subject matter does not claim to treat chronic venous insufficiency or varicose veins. Instead, one of the objects of the present subject matter is to provide relief to people suffering from chronic venous insufficiency by controlling its symptoms by providing medical-grade compression socks, stockings, and/or sleeves adapted and configured to make it tolerable and acceptable for people who may otherwise refuse to wear them despite a recommendation from a physician.

After endovenous radiofrequency or laser-ablation treatment, treated patients are advised to wear compression stockings primarily to prevent the risk of thrombus extending from treated saphenous veins into a deep vein. Thrombus extension, a known complication of endovenous ablation procedures, is commonly referred to as endothermal heat-induced thrombosis (“EHIT”). The present subject matter, which is configured to deliver medical-grade pressure along treated patient saphenous veins, prevents the risk of EHIT and makes it easier for patients to wear compression stockings during recovery.

Since saphenous veins are typically treated by damaging the endothelial wall of a patient using thermal heat, there is an associated inherent risk because whenever the body of a patient senses that the superficial veins have already been obliterated and are no longer functioning, a blood clot will often form in the treated veins within 24 to 72 hours.

My linear pads, when applied along treated saphenous veins, are configured and dimensioned to ensure that diameters of varicose or dilated veins are reduced. By doing so, my linear pads will minimize the formation of blood clots in the treated veins of the patient. Another important result of using my pads is to ensure blood clots remain stable. Newly formed blood clots, referred to as acute blood clots, are soft in texture. However, my linear pads, extend into tiny areas and do not move. Thus, the present subject matter not only aids in recovery and reduces inflammation; it also prevents risk of EHIT as well.

### SUMMARY

Embodiments of the present subject matter are directed to compression socks and stockings designed and adapted to be more comfortable to wear each day and to be easier for a patient to put on in comparison to conventional socks and stockings. They apply surface pressure to select leg regions to decompress abnormal saphenous veins present there. The medical-grade compression stockings of the present subject matter are manufactured from fabric and elastic materials having a strength effective to provide a surface pressure of about 15-20 mm Hg circumferentially inwardly around a leg of a person having abnormal saphenous veins. My compression stockings, made to include linear pads of the same or similar materials, apply constant pressure to saphenous veins.

The medical-grade compression legwear of the present subject matter includes a thigh-high medical-grade compression stocking embodiment as well as a knee-high medical-grade compression sock embodiment. For the knee-high compression sock embodiment, two linear pads, each measuring about 4.0 cm wide and about 6.0 mm thick extend from the knee to the ankle of a patient, medially and posteriorly. For the thigh-high compression stocking embodiment, in addition to the two linear pads noted above for the knee-high compression sock embodiment, the knee-high compression sock embodiment includes an additional linear pad with a width of about 5.0 cm and a thickness of about 8.0 mm extending from the thigh proximally to the knee medially, and another along the thigh proximally (about 10 cm in length) and anteriorly. Specifications for the knee-high sock embodiment can be used for lower sections of the thigh-high compression stockings.

The illustrated embodiment of the present subject matter, directed to 15-20 mm Hg compression legwear designed to deliver a surface pressure of about 20-30 mm Hg to skin under which there are saphenous veins, does so with integrated linear pads. The pads, made of the same fabric and elastic material as the sock, are designed, sized, adapted, and configured to target locations of saphenous veins under the skin. For purposes of this patent specification, the term “pad” when used in connection with “a linear pad” shall be understood to mean a relatively thick piece of soft material that, by applying pressure to a surface, presses down upon a portion of such surface. After a pad is applied to a leg, e.g., the pad changes the surface contours of the skin. An applied pad increases the pressure on the skin and decreases volume within surrounding soft tissue. As pads increase pressure above abnormal saphenous veins from about 15-20 mm Hg to 20-30 mm Hg, contacted varicose vein diameters reduce back to their normal size. Once this is accomplished, refluxing valves in saphenous veins can work normally again.

Another embodiment of the present subject matter is directed to medical-grade compression sleeves to be worn on the legs of a person having abnormal saphenous veins. Compression sleeves, of the present subject matter, have been found to be easier for a patient put on his or her legs than conventional compression sleeves and are more comfortable to wear. My medical-grade compression sleeves apply surface pressure to select leg regions to decompress abnormal saphenous veins present there. My medical-grade compression sleeves are made from fabric and elastic materials having a strength effective to provide a surface pressure of about 15-20 mm Hg both circumferentially and inwardly around a leg of a person having abnormal saphenous veins. My medical-grade compression sleeves are made to include linear pads of the same or similar materials, for applying constant pressure to saphenous veins. Embodiments of the present subject matter may add an additional surface pressure of about 15-25 mm Hg or about 20-30 mm Hg or about 25-35 mm Hg to the skin of a patient, under which there are saphenous veins.

While my compression sock and stocking embodiments may not be suitable for people with lymphedema and other non-vascular-related causes of swelling in feet, my compression sleeve embodiment is a better therapy solution for people who are suffering from chronic venous insufficiency for several reasons. Sleeves are easier to put on, more comfortable to wear, are less costly to manufacture, and linear pads are easier to attach.

The compression sleeves of the present subject matter include a thigh-high variety and a knee-high variety. For the knee-high variety, two linear pads, each measuring about 4.0 cm wide and 6.0 mm thick, extend from the knee to the ankle, medially and posteriorly. In addition, for the thigh-high variety, a linear pad having a width of about 5.0 cm and a thickness of 8.0 mm extends from the proximal thigh to the knee medially, and another pad extends along the proximal thigh (about 10 cm in length) anteriorly. Specifications for knee-high compression sleeve varieties may also be used for lower sections of thigh-high varieties. This compression sleeve embodiment, for 15-20 mm Hg sleeves designed and adapted to deliver a surface pressure of, e.g., about 20-30 mm Hg, to the skin under which there are saphenous veins, does so with integrated linear pads. Such pads, made of the same fabric and elastic material, are adapted and configured to target locations of saphenous veins under the skin of a leg of a person having abnormal saphenous veins.

Yet another embodiment of the present subject matter is directed to medical-grade compression legwear which includes a zipper substantially along an entire length thereof.

A compression sleeve embodiment of the present subject matter comprises a compression body, at least one elongated pad secured to an inner surface portion of the body, and a zipper (or slide fastener). The body is adapted and configured to be worn by a person having at least one varicose vein. The body has an upper edge margin and a lower edge margin spaced from the upper edge margin. The body defines a linear opening extending along an imaginary line oriented between the upper and lower edge margins.

The at least one elongated pad is secured to an inner surface portion of the body. The pad overlays a major portion of at least one varicose vein of a person wearing the sleeve. The pad has opposite end portions. One of the end portions is located proximate to one of the upper and lower edge margins. The other end portion is proximate to the other one of the upper and lower edge margins. The pad is adapted and configured to exert a compression of about 20-30 mm Hg to the major portion of said varicose vein.

The zipper (or slide fastener) is secured to the body, overlays the linear opening, and is movable between a first position to enable a person to remove the sleeve and a second position spaced from the first position for causing the pad to exert a compression of about 20-30 mm Hg to the major portion of said varicose vein of a wearer of the sleeve.

A compression sock embodiment of the present subject matter comprises a compression body, at least one elongated pad secured to an inner surface portion of the body, and a zipper (or slide fastener). The body is adapted and configured to be worn by a person having at least one varicose vein. The body defines an upper portion, located proximate to a knee of a wearer of the sock. The body also defines a lower portion spaced from the upper portion. The lower portion of the body is sized and configured to fit a foot, of a wearer of the sock. The compression body also defines a linear opening extending along an imaginary line oriented between the knee and the foot of the wearer of the sock.

The at least one elongated pad is secured to an inner surface portion of the body. The elongated pad overlays a major portion of at least one varicose vein of a person wearing the sock. The elongated pad has opposite end portions. One of the end portions is located proximate to one of (the knee and the foot of the wearer of the sock). The other end portion is located proximate to the other one (of the knee and foot). The at least one elongated pad is sized, adapted, and configured to exert a compression of about 20-30 mm Hg to the major portion of the at least one varicose vein of the wearer of the sock.

The zipper (or slide fastener) is secured to the body, overlays the linear opening, and is movable between a first position to enable a person to remove the sock and a second position spaced from the first position for causing the pad to exert a compression of about 20-30 mm Hg to the major portion of said varicose vein of the wearer of the sock.

A compression stocking embodiment of the present subject matter comprises a compression body, at least one elongated pad secured to an inner surface portion of the body, and a zipper (or slide fastener). The body is dimensioned and configured to be worn by a person having at least one varicose vein. The body defines an upper portion dimensioned and configured to fit a thigh (of a wearer of the stocking). The body defines a lower portion spaced from the upper portion. The lower portion is sized and configured to fit a foot of a wearer of the stocking. The body defines a linear opening extending along an imaginary line that is oriented between the thigh and foot of a wearer of the stocking.

The at least one elongated pad is secured to an inner surface portion of the body.

The pad overlays a major portion of at least one varicose vein of a person wearing the stocking. The elongated pad has opposite end portions. One of the end portions is located proximate to one of the thigh and the foot of the wearer of the stocking. The other end portion is located proximate to the other one (of the thigh and the foot). The at least one elongated pad is adapted and configured to exert a compression of about 20-30 mm Hg to the major portion of the at least one varicose vein of the wearer of the stocking.

The zipper (or slide fastener) is secured to the body, overlays the linear opening, and is movable between a first position to enable a person to remove the stocking and a second position spaced from the first position for causing the pad to exert a compression of about 20-30 mm Hg to the major portion of the varicose vein of a wearer of the stocking.

### BRIEF DESCRIPTION OF THE DRAWINGS

FIG. 1 depicts an embodiment of a knee-high compression sock of the present subject matter on a left leg, medial side, of a typical wearer of the compression sock.

FIG. 2 depicts an embodiment of a knee-high compression sock of the present subject matter on a right leg, medial side, of a typical wearer of the compression sock.

FIG. 3 depicts an embodiment of a thigh-high compression stocking of the present subject matter on a left leg, medial side, of a typical wearer of the compression stocking.

FIG. 4 depicts an embodiment of a thigh-high compression stocking of the present subject matter on a right leg, medial side, of a typical wearer of the compression stocking.

FIG. 5 depicts an embodiment of a thigh-high compression sleeve of the present subject matter on a right leg, medial side, of a typical wearer of the compression sleeve.

FIG. 6 depicts an embodiment of a knee-high compression sleeve of the present subject matter on a right leg, medial side, of a typical wearer of the compression sleeve.

FIG. 7 depicts an embodiment of a thigh-high compression sleeve of the present subject matter on a left leg, medial side, of a typical wearer of the compression sleeve.

FIG. 8 depicts an embodiment of a knee-high compression sleeve of the present subject matter on a left leg, medial side, of a typical wearer of the compression sleeve.

FIG. 9 presents an embodiment of a portion of a pad of the present subject matter.

FIG. 10 depicts an embodiment of a knee-high compression sock of the present subject matter on a right leg of a typical wearer. The sock includes a posterior-mounted linear pad, a lateral-mounted zipper, and the medial-mounted linear pad shown in FIG. 2.

FIG. 11 depicts an embodiment of a knee-high compression sock of the present subject matter on a left leg of a typical wearer. The sock includes a posterior-mounted linear pad, a lateral-mounted zipper, and the medial-mounted linear pad shown in FIG. 1.

FIG. 12 illustrates an embodiment of a thigh-high compression stocking of the present subject matter on a right leg. The stocking includes a posterior-mounted linear pad, a lateral-mounted zipper, and several medial-mounted linear pads shown in FIG. 4.

FIG. 13 illustrates an embodiment of a thigh-high compression stocking of the present subject matter worn on a left leg. The stocking includes a posterior-mounted linear pad, a lateral-mounted zipper, and several medial-mounted linear pads shown in FIG. 3.

Throughout the figures—and the detailed description which follows—I shall use similar reference numerals, to refer to similar components of the present subject matter.

### DETAILED DESCRIPTION

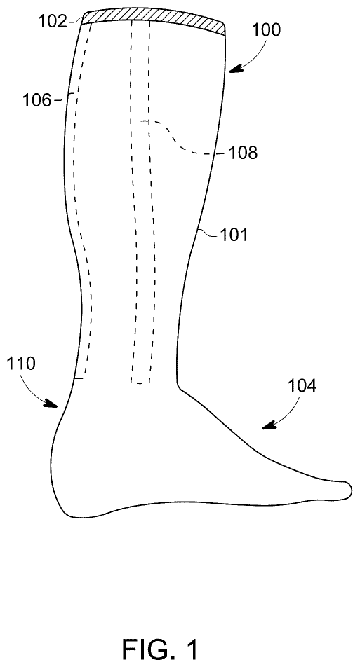

Compression socks and stockings (FIGS. 1-4) of the present subject matter are designed, dimensioned, and configured to use a technique called “targeted compression.”

With targeted compression, an entire leg doesn't need to be tightly compressed. Instead, only locations where the great and small saphenous veins are located are firmly compressed by linear pads sewn to compression footwear of the present subject matter.

For purposes of the present subject matter, the term “pad” shall be understood to mean a relatively thick piece of relatively soft material adapted and configured to change the shape of something. Therefore, when a pad is applied to a leg, the pad changes the surface contour of the skin, increasing pressure in surrounding soft tissue and decreasing its volume. When applied thusly, a pad reduces the superficial vein diameter and when compression socks and stockings are worn, pressure at an arc length of a leg not covered by pads is 15-20 mm Hg, while pressure at an arc length of a leg covered by a pad of the present subject matter increases to about 20-30 mm Hg, as additional material pushes skin and soft tissue. A pressure of, for example, about 20-30 mm Hg decompresses refluxing great and small saphenous veins, by reducing their size and enabling associated valves to work properly again. As a result, venous hypertension in lower legs is relieved.

The concept of targeted compression is based on a procedure involving ultrasound probe pressure, whereby varicose great and small saphenous veins decompress when adequate pressure is applied to medial and posterior sides of legs using an ultrasound probe. Linear pads, designed into my compression footwear, function like ultrasound probes, to constantly apply pressure at locations where abnormal saphenous veins are.

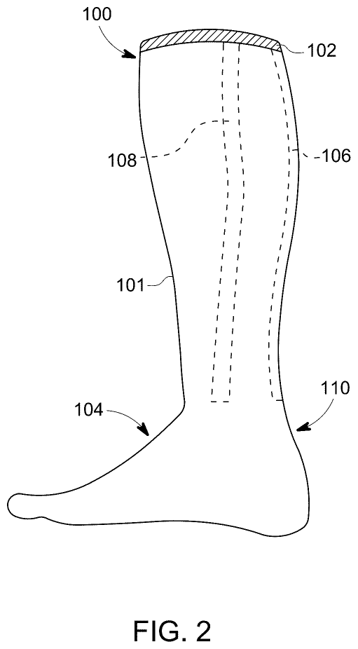

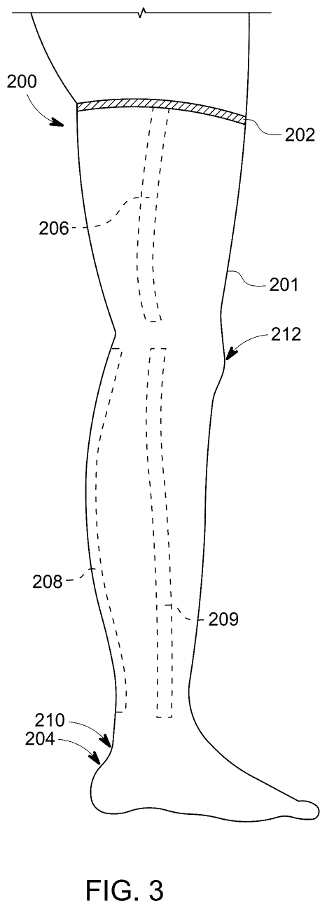

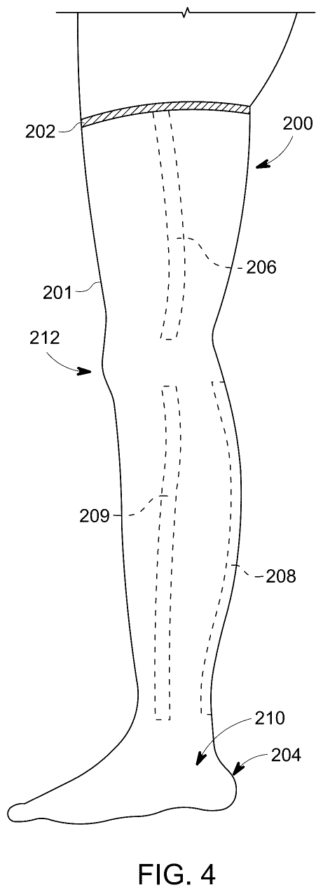

FIGS. 1-4 show embodiments of the present subject matter. FIGS. 1 and 2 depict embodiments of a knee-high compression sock of the present subject matter on a left leg (FIG. 1) and right leg (FIG. 2), medial side, of a typical compression sock wearer. FIGS. 3 and 4 depict embodiments of a thigh-high compression stocking of the present subject matter on a left leg (FIG. 3) and right leg (FIG. 4), medial side, of a typical stocking wearer.

Referring to FIGS. 1 and 2, a knee-high compression sock 100 embodiment of the present subject matter includes an upper margin 102, a lower region 104, and at least two linear pads 106 and 108 fixed to inner surface portions of the compression sock 100 and extending from the upper margin 102 by a knee (not shown) to a lower region 104 below the ankle 110. Lower region 104 is sized and configured to snuggly receive a foot of a person. The linear pads 106, 108 are elongated, vertically oriented, and spaced apart. Trailing edge-mounted linear pads 106 are vertically arranged along a wearer calf and oriented about ninety angular degrees (90°) about a leg (of a wearer of compression sock 100) from an associated medial side-mounted pad 108. Each linear pad 106 and 108 has a width ranging from about 1 cm to about 5 cm, from about 2 cm to about 5 cm, or from about 3 cm to about 5 cm and has a thickness ranging from about 1 mm to about 8 mm, from about 2 mm to about 8 mm, from about 3 mm to about 8 mm, or from about 4 mm to about 8 mm, or from about 5 mm to about 8 mm, or from about 6 mm to about 8 mm.

The sock 100 of the present subject matter includes a compression body 101 made of spandex, a versatile fabric capable of being blended with other materials including but not limited to cotton, polyester, nylon, and combinations thereof for thereby providing a variety of fabrics and fabric blends for the present subject matter. For instance, a fabric blend ranging from about 23 weight-percent (wt.-%) spandex and 77 wt.-% nylon to about 27 wt.-% spandex and 73 wt.-% nylon is believed suitable for the present subject matter.

Referring next to FIGS. 3 and 4, an embodiment of a thigh-high compression stocking 200 of the present subject matter includes an upper margin 202, a lower region 204, and at least three linear pads 206, 208, 209 secured to select inner surface portions of the compression stocking 200. A shorter linear pad 206 and a longer linear pad 209 are secured to inner surface portions of the stocking 200 at an upper medial location and at a lower medial location respectively. A linear pad 208 is secured to an inner surface portion of the stocking 200 at an upper frontal location and at a lower trailing location respectively. The linear pad 208 secured to the inner surface portion of the stocking 200 at the lower trailing location that extends along a calf of a wearer of the stocking 200. The lower location is located proximate to an ankle 210 of the stocking wearer; and the lower region 204 is dimensioned and configured to snuggly receive a foot of the stocking wearer.

The upper pad 206, positioned along a thigh of a wearer of a stocking 200, has a width ranging from about 1 cm to about 5 cm, or from about 2 cm to about 5 cm, or from about 3 cm to about 5 cm, and has a thickness ranging from about 1 mm to about 8 mm, from about 2 mm to about 8 mm, or from about 3 mm to about 8 mm, or from about 4 mm to about 8 mm, or from about 5 mm to about 8 mm, or from about 6 mm to about 8 mm.

Each lower pad 208, 209 —positioned along a calf of a wearer of a stocking 200, with lower pads 208 and 209 each having substantially the same length and extending between an ankle 210 and a knee 212 of a wearer of stocking 200—has a width ranging from about 1 cm to about 4 cm, from about 2 cm to about 4 cm, or from about 3 cm to about 4 cm, and has a thickness ranging from about 1 mm to about 6 mm, from about 2 mm to about 6 mm, from about 3 mm to about 6 mm, or from about 4 mm to about 6 mm.

The stocking 200 of the present subject matter includes a compression body 201 made of spandex, a versatile fabric able to be blended with other materials including but not limited to cotton, polyester, nylon, and combinations thereof for thereby providing a variety of fabrics and fabric blends for the present subject matter. For instance, a fabric blend ranging from about 23 weight-percent (wt.-%) spandex and 77 wt.-% nylon to about 27 wt.-% spandex and 73 wt.-% nylon is believed suitable for the present subject matter.

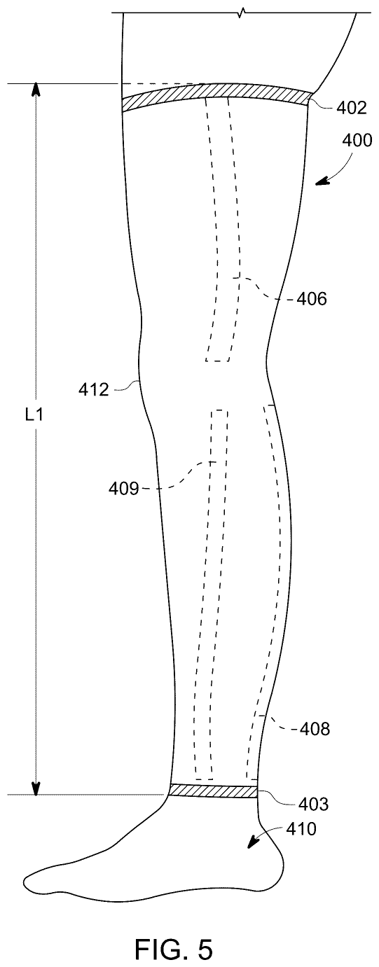

Compression sleeves (FIGS. 5-8) of the present subject matter are also designed, sized, adapted, and configured to use the “targeted compression” technique noted above.

For purposes of the present subject matter, the term “pad” shall be understood to mean a relatively thick piece of relatively soft material sized, adapted, and configured to change the shape of a surface portion of a leg to which the pad is applied. When a pad is applied to a leg, the pad changes the surface contour of a portion of a leg to which the pad is applied, thereby increasing pressure within surrounding soft tissue and decreasing its volume. When applied thusly, a pad reduces the superficial vein diameter and when compression sleeves are worn, pressure at the arc length of a leg not covered by pads is 15-20 mm Hg, while pressure at the arc length of a leg covered by a pad of the present subject matter, is increased to about 20-30 mm Hg, as the additional material pushes the skin and soft tissue. A pressure of, for example, about 20-30 mm Hg decompresses the refluxing great and small saphenous veins, by reducing their sizes and enabling their valves to work properly again. As a result, venous hypertension in lower legs is relieved.

The concept of targeted compression is based on a procedure involving ultrasound probe pressure, whereby the varicose great and small saphenous veins will decompress when adequate pressure is applied to the medial and posterior sides of legs using an ultrasound probe. Linear pads incorporated into my compression sleeve design function as ultrasound probes, to constantly apply pressure where abnormal saphenous veins are.

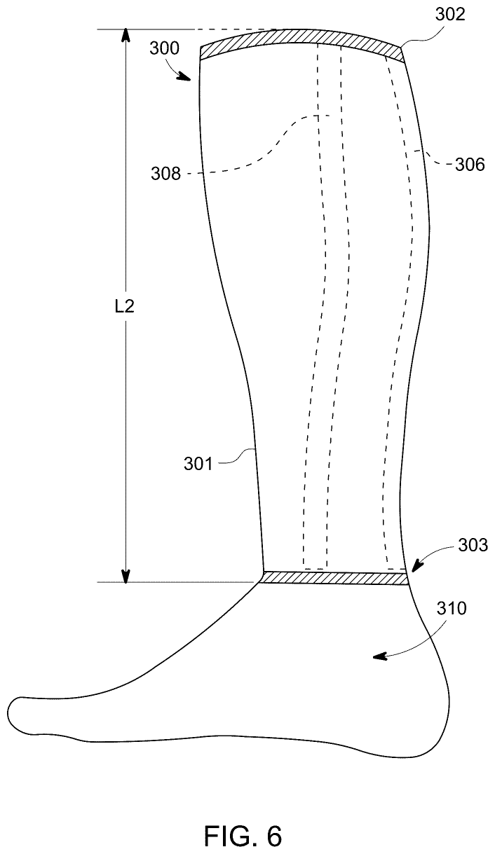

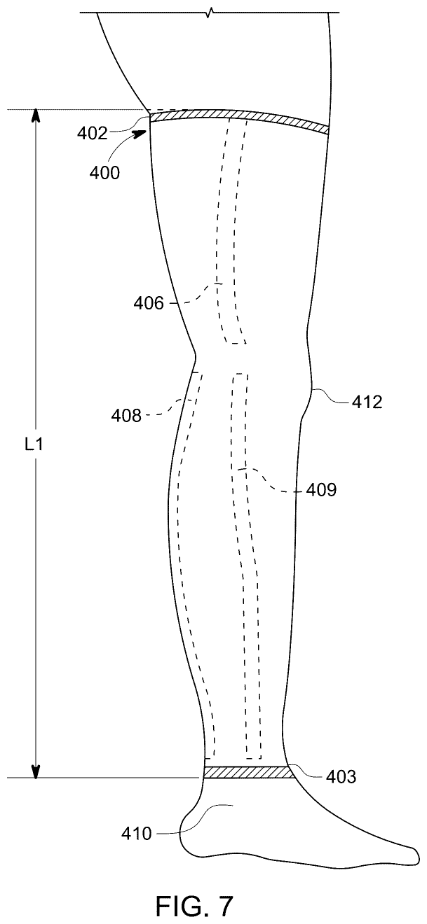

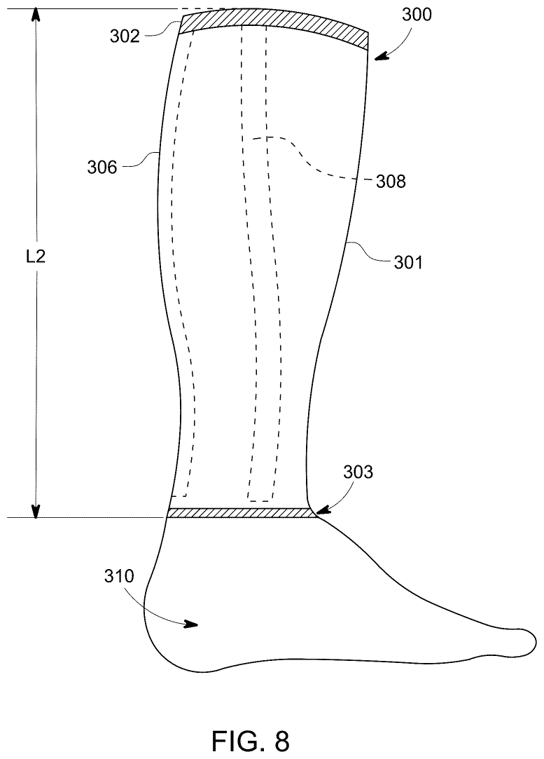

FIGS. 5-8 also illustrate embodiments of the present subject matter. FIGS. 6, 8 are examples of the knee-high compression sleeves of the present subject matter on a right leg (FIG. 6) and on a left leg (FIG. 8), medial side, of a typical compression sleeve wearer. In addition, FIGS. 5, 7 are examples of the thigh-high compression sleeve of the present subject matter on a right leg (FIG. 5) and left leg (FIG. 7), medial side, of a typical wearer.

Referring to FIGS. 6 and 8, an embodiment of a knee-high compression sleeve 300 of the present subject matter includes an upper margin 302, a lower margin 303, and at least two linear pads 306 and 308 secured to inner surface portions of the compression sleeve 300 and extending from the upper margin 302 to the lower margin 303 adjacent to an ankle 310 of a wearer of the knee-high compression sleeve 300. Depending upon the physical dimensions of a person, a knee-high compression sleeve 300 of the present subject matter may have a length (“L2”) ranging from about 10 cm to about 80 cm or from about 15 cm to about 75 cm or from about 20 cm to about 70 cm or from about 25 cm to about 65 cm or from about 30 cm to about 60 cm or from about 35 cm to about 55 cm.

The linear pads 306 and 308 of the present subject matter are elongated. They are substantially vertically oriented when worn and are spaced apart. Trailing edge-mounted portions of the elongated pad 306 is essentially vertically arranged along a wearer calf and is oriented about 90° from an associated medial pad 308. Each pad 306, 308 has a width ranging from about 1 cm to about 5 cm, from about 2 cm to about 5 cm, or from about 3 cm to about 5 cm and has a thickness ranging from about 1 mm to about 8 mm, from about 2 mm to about 8 mm, or from about 3 mm to about 8 mm, or from about 4 mm to about 8 mm, or from about 5 mm to about 8 mm, or from about 6 mm to about 8 mm.

The sleeve 300 of the present subject matter includes a compression body 301 made of spandex, a versatile fabric able to be blended with other materials including but not limited to cotton, polyester, nylon, and combinations thereof for thereby providing a variety of fabrics and fabric blends for the present subject matter. For instance, a fabric blend ranging from about 23 weight-percent (wt.-%) spandex and 77 wt.-% nylon to about 27 wt.-% spandex and 73 wt.-% nylon is believed suitable for the present subject matter.

Referring to FIGS. 6 and 8, a circumferential distance between vertically oriented linear pads 306 and 308 may vary within a range of between about 5 cm to about 30 cm, about 7 cm to about 25 cm, about 9 cm to about 20 cm, or about 11 cm to about 15 cm.

Referring next to FIGS. 5 and 7, embodiments of a thigh-high compression sleeve 400 of the present subject matter include an upper margin 402, a lower margin 403, and at least three linear pads 406, 408, 409 fixed to inner surface portions of the compression sleeve 400 and extending either from the upper margin 402 or to the lower margin 403. Thigh-high compression sleeve 400 has a length (“L1”) ranging from about 10 cm to about 150 cm, from about 20 cm to about 140 cm, from about 30 cm to about 130 cm, from about 35 cm to about 125 cm, from about 40 cm to about 120 cm, from about 50 cm to about 115 cm, from about 55 cm to about 110 cm, or from about 60 cm to about 105 cm.

Each upper pad 406 of the thigh-high compression sleeve 400 is elongated, oriented substantially vertically, and positioned on a thigh of a wearer. Pad 406 has a width ranging from about 1 cm to about 5 cm, or from about 2 cm to about 5 cm, or from about 3 cm to about 5 cm and has a thickness ranging from about 1 mm to about 8 mm, from about 2 mm to about 8 mm, or from about 3 mm to about 8 mm, or from about 4 mm to about 8 mm, or from about 5 mm to about 8 mm, or from about 6 mm to about 8 mm.

Each lower pad 408 and 409—positioned along a calf of a wearer of sleeve 400, with lower pads 408 and 409 each having substantially the same length and extending between an ankle 410 and a knee 412 of a wearer of sleeve 400—has a width that ranges from about 1 cm to about 4 cm, from about 2 cm to about 4 cm, or from about 3 cm to about 4 cm, and has a thickness ranging from about 1 mm to about 6 mm, from about 2 mm to about 6 mm, from about 3 mm to about 6 mm, or from about 4 mm to about 6 mm.

The sleeve 400 of the present subject matter includes a compression body portion 401 made of spandex, a versatile fabric able to be blended with other materials including but not limited to cotton, polyester, nylon, and combinations thereof for thereby providing a variety of fabrics and fabric blends for the present subject matter. For instance, a fabric blend ranging from about 23 weight-percent (wt.-%) spandex and 77 wt.-% nylon to about 27 wt.-% spandex and 73 wt.-% nylon is believed suitable for the present subject matter.

Referring to FIG. 5, a circumferential distance between vertically oriented linear pads 408 and 409 may vary within the range of between about 5 cm to about 30 cm, about 7 cm to about 25 cm, about 9 cm to about 20 cm, or about 11 cm to about 15 cm.

Embodiments of the present subject matter —including the knee-high compression sock 100, the thigh-high compression stocking 200, the knee-high compression sleeve 300, and the thigh-high compression sleeve 400 of the present subject matter—resolve the most common complaints of traditional compression stocking wearers: i.e., traditional compression stockings are said to be difficult to put on and too tight when worn. So how comfortable are the 15-20 mm Hg compression footwear with linear pads—of the present subject matter—in comparison to traditional 20-30 mm Hg compression socks/stockings? According to the Center for Disease Control (“CDC”) and Prevention, the average calf circumference is 39.37 centimeters (15.5 inches) for male, and 38.1 cm (15.0 in) for female; overall mean calf circumference is 38.74 cm (15.25 in). Conventional knee-high padded compression stockings use a total width of about 8.0 cm for two pads. Pressure distribution in the calf with conventional knee-high compression stockings is as follows:
Unpadded parts of the stocking: 15-20 mm Hg around 28.74 cm of arc length.
Padded parts of the stocking have: 20-30 mm Hg around 8.0 cm of arc length.

On a “personal comfort” (i.e., subjective) basis, it is believed a person of ordinary skill in the art (“POSITA”) would likely conclude that knee-high 15-20 mm Hg compression stockings including the linear pads—of the present subject matter—are about 79 % more comfortable in the calves, compared to traditional 20-30 mm Hg compression stockings.

Based on body measurement data from the US Department of Health Statistics, the average circumference of a thigh of a man is 53.8 cm (21.2 inches), and for a female is 52.9 cm (20.8 inches). The overall mean thigh circumference is 53.35 cm (21 inches). The pressure distribution in the thigh with thigh-high compression stockings is as follows:
Unpadded parts of the stocking: 15-20 mm Hg around 43.35 cm of arc length.
Padded parts of the stocking: 20-30 mm Hg around 10 cm of arc length.

Hence, it is believed a person of ordinary skill in the art (“POSITA”) would likely conclude thigh-high 15-20 mm Hg padded compression stockings are about 81 % more comfortable in upper legs, compared to traditional 20-30 mm Hg compression stockings.

There are at least two ways to attach elongated pads to my compression legwear. One way involves using commercially available elastic and/or flexible glues or adhesives. Illustrative examples include U.S. Pat. Nos. 3,515,773; 3,671,301; and 3,756,848 to Dahl; and U.S. Pat. No. 3,940,513 to Anderson—all of which are hereby incorporated by reference in their entirety. Another procedure involves permanently securing the elongated pads to my compression legwear by sewing or stitching pads to a compression sock, stocking, or sleeve by using a preselected material of predetermined strength, e.g., nylon or polyester and straight stitching, which I prefer doing to secure the pads to the compression legwear.

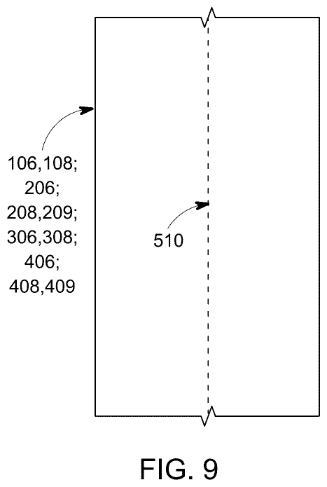

FIG. 9 presents a portion of a pad of the present subject matter. Elongated pads 106, 108; 206, 208, 209; 306, 308; 406, 408, 409 may be produced with a partial or complete slit 510 along a length of a longitudinal pad from one end portion to the other. Pads may be secured to my compression legwear by sewing along slit 510 from one end portion to the other. After being secured, pad sections may be fused along the slit 510 using examples of commercially available elastic/flexible glues or adhesives listed above.

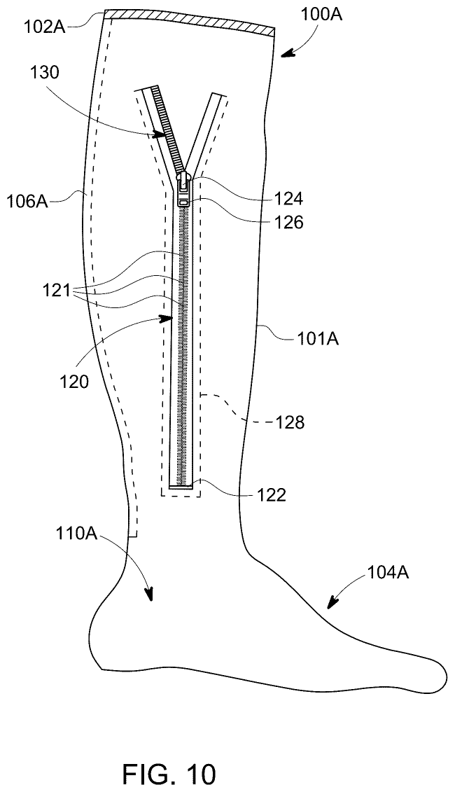

FIGS. 10-13 depict another embodiment of medical-grade compression socks and stockings of the present subject matter where each sock and stocking includes a zipper along most of its length and linear pads for pre-and post-operative endovenous ablation.

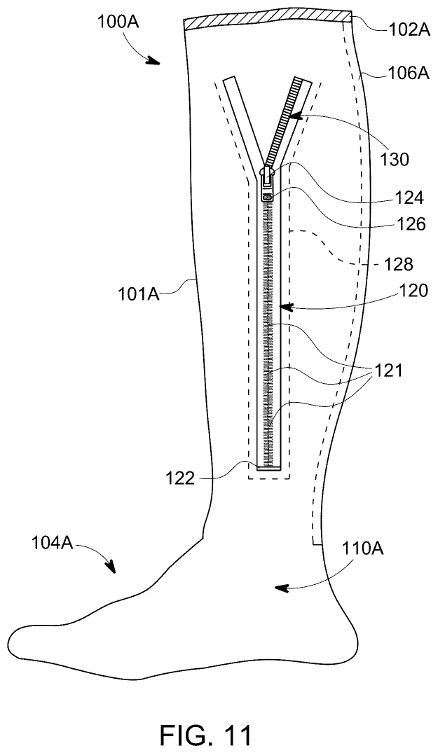

FIGS. 10, 11 illustrate embodiments of a knee-high compression sock 100A of the present subject matter on a right leg (FIG. 10) and left leg (FIG. 11) of a typical wearer of the sock 100A. Each sock 100A has a compression body 101A, an upper margin 102A (that may include elastic material to keep sock 100A around a leg and below a knee), a posterior linear pad 106A (FIGS. 10, 11), a medial linear pad 108 (shown only in FIGS. 1, 2), and a zipper 120 (FIGS. 10, 11). The posterior linear pad 106A, medial linear pad 108, and zipper 120 are each vertically oriented substantially along a length of sock 100A. The zipper 120 includes a plurality of engageable teeth 121 along its length, a stop 122 along a lower end portion thereof, and a slide 124 used to engage and disengage the teeth 121.

Attached to the slide 124 is a pull 126 which a wearer of the sock 100A may use to move slide 124 between stop 122 and the opposite end of zipper 120 for the purpose of bringing the teeth 121 together, to close the zipper 120. A backside of the entire length of zipper 120 includes a skin protector 130, sized, designed, adapted, and configured to protect the skin of a leg of a wearer of sock 100A from the teeth 121 of the zipper 120. A backside portion (not shown) of zipper 120, including the skin protector 130, are depicted (by a broken line 128) as securely sewn to the compression body 101A of the sock 100A. The zipper 120 of the knee-high compression sock 100A embodiment of the present subject matter (FIGS. 10, 11) extends from a point located below the upper margin 102A to stop 122 at the lower end portion of zipper 120 near an ankle 110A of a wearer. A lower region 104A of sock 100A is sized and configured to snuggly receive a foot of the wearer.

The posterior-mounted linear pads 106A of the knee-high compression socks 100A, when worn on the right (FIG. 10) and left (FIG. 11) legs of a patient, are vertically arranged along an associated calf of the patient wearing the socks 100A, and are oriented about ninety angular degrees (90°) about the legs of a wearer of the compression socks 100A, in relation to the medial-mounted linear pads 108 (shown only in FIGS. 1 and 2).

Both linear pads 106A and 108 are sized, adapted, and configured to exert a pressure of about 20-30 mm Hg along a varicose vein of a wearer of the knee-high compression socks 100A (FIGS. 10, 11). The compression body 101A of each left and right sock 100A defines a “cut” or linear opening (not shown) located either adjacent to or overlaying a major portion of a varicose vein of a wearer of the compression sock 100A.

Zipper 120 (or “slide fastener” or “fastener”) overlays the “cut” or linear opening (not visible) defined by body 101A of the knee-high compression socks 100A. Each linear pad 106A (FIGS. 10, 11) and 108 (shown only in FIGS. 1, 2) has a width ranging from about 1 cm to about 5 cm, from about 2 cm to about 5 cm, or from about 3 cm to about 5 cm. Also, each linear pad 106A, 108 has a thickness ranging from about 1 mm to about 8 mm, from about 2 mm to about 8 mm, from about 3 mm to about 8 mm, from about 4 mm to about 8 mm, from about 5 mm to about 8 mm, or from about 6 mm to about 8 mm.

The knee-high compression sock 100A of the present subject matter includes a body portion 101A made of spandex, a versatile fabric capable of being blended with other materials including but not limited to cotton, polyester, nylon, and combinations thereof, for providing a variety of fabrics and fabric blends. For instance, a fabric blend that ranges from about 23 weight-percent (wt.-%) spandex and 77 wt.-% nylon to about 27 wt.-% spandex and 73 wt.-% nylon is believed suitable for the present subject matter.

For this embodiment, located immediately beneath the teeth 121 of the zipper 120 (in the direction of skin on a leg of a wearer of the compression socks 100A), the body portion 101A of the socks 100A (FIGS. 10, 11) define a “cut” or linear opening (not visible) substantially equal to the length of the zipper 120, for enabling wearers of each knee-high compression sock 100A to easily put it on their legs and easily remove it from their legs.

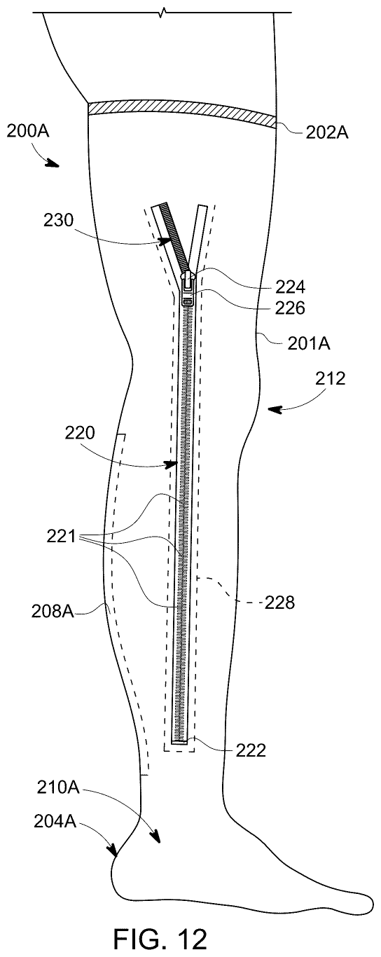

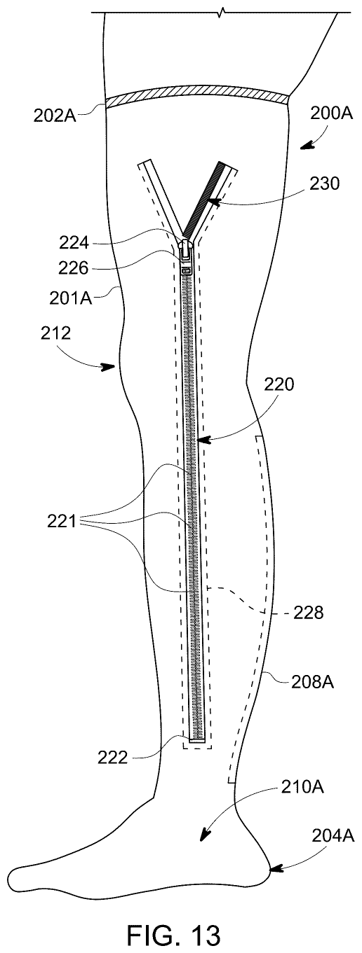

FIGS. 12, 13 depict an embodiment of a thigh-high compression stocking 200A of the present subject matter on a right (FIG. 12) and left (FIG. 13) leg of a typical wearer of stocking 200A. Each stocking 200A includes a compression body 201A, an upper margin 202A, a posterior linear pad 208A, vertically oriented and spaced apart upper and lower medial pads 206 and 209 (shown only in FIGS. 3, 4), and a zipper 120 (FIGS. 12, 13). The posterior linear pad 208A and the zipper 220 are each vertically oriented substantially long the length of each thigh-high compression stocking 200A. The zipper 120 includes a plurality of engageable teeth 221 along its length, a stop 222 secured fixed to a lower end portion of zipper 220, and a slide 224 for engaging and disengaging the plural teeth 221.

Attached to the slide 224 is a pull 226, which a wearer of the stocking 200A may use to move slide 224 between the stop 222 and the opposite end of zipper 220 for the purpose of bringing the teeth 221 together, to close zipper 220. A backside of the entire length of zipper 220 includes a skin protector 230, dimensioned, designed, adapted, and configured to protect the skin of a leg of a wearer of stocking 200A from the teeth 221 of zipper 220. A backside portion (not shown) of zipper 220 including skin protector 230 are shown (by a broken line 228) as securely sewn to the compression body 201A of stocking 200A. Zipper 220 of the thigh-high compression stocking 200A embodiment of the present subject matter (FIGS. 12, 13) extends from a point located below the upper margin 202A to stop 222 at the lower end portion of zipper 220 near an ankle 210A of a wearer. A lower region 204A of stocking 200A is sized and shaped to snuggly receive a foot of the wearer.

The posterior-mounted linear pads 208A of the thigh-high compression stockings 200A, when worn on the right (FIG. 12) and on the left (FIG. 13) leg of a patient or another person, are vertically arranged generally along associated calves of the patient or another person wearing the thigh-high compression stockings 200A, and are oriented about ninety angular degrees (“90°”) about the legs of such wearer of the stockings 200A in relation to the upper and the lower vertically spaced apart, medial-mounted linear pads 206 and 209.

Each linear pad 206, 208A, and 209 is dimensioned and configured to exert a pressure of about 20-30 mm Hg along a varicose vein of a wearer of the thigh-high compression stocking 200A (FIGS. 12, 13). The body portion 201A of each left and right thigh-high stocking 200A defines a linear opening (not shown) located either adjacent to or overlaying a major portion of a varicose vein of a wearer of compression stocking 200A.

The zipper 220 (or slide fastener 220) overlays a substantially linear opening defined by the compression body 201A of the thigh-high compression stocking 200A (FIGS. 12, 13). Each of the upper medial-mounted linear pad 206, the posterior-mounted linear pad 208A, and the lower medial-mounted linear pad 209 (shown only in FIGS. 3 and 4) has a width that ranges from about 1 centimeter (“cm”) to about 5 cm, or from about 2 cm to about 5 cm, or from about 3 cm to about 5 cm. Furthermore, each linear pad 206, 208A, and 209 has a thickness ranging from about 1 millimeter (“mm”) to about 8 mm, from about 2 mm to about 8 mm, from about 3 mm to about 8 mm, from about 4 mm to about 8 mm, from about 5 mm to about 8 mm, or from about 6 mm to about 8 mm.

The stocking 200A of the present subject matter includes the body portion 201A manufactured of spandex, a versatile fabric capable of being blended with other materials including but not limited to cotton, polyester, nylon, and combinations thereof, to thereby provide various fabrics and fabric blends for the present subject matter. For instance, a fabric blend ranging from about 23 weight-percent (wt.-%) spandex and 77 wt.-% nylon to about 27 wt.-% spandex and 73 wt.-% nylon is suitable for the present subject matter.

In this embodiment of the present subject matter, stocking 200A includes a zipper 220 shown securely sewn to the body portion 201A of stocking 200A via broken line 228. Zipper 220 includes a stop 222 at one end portion, near an ankle 210. A skin protector 230 at the opposite end portion of zipper 220 is located near the upper margin 202A. The zipper 220 includes a pair of interlocking teeth 221 oriented along its length. The zipper 220 also includes a slide 224 adapted to slide along the teeth 221, and a pull 226 that is secured to the slide 224 for enabling a user to join the teeth 221 when pulling the slide 224 across skin protector 230 toward the upper margin 202A. Moreover, slide 224 also enables the user to separate the teeth 221 when slide 224 is pulled toward the stop 222.

Zippers (also called “slide fasteners” or simply “fasteners” by persons of ordinary skill in the art) have been well known for many years. See, e.g., U.S. Pat. No. 1,219,881 to Sundback; U.S. Pat. No. 1,948,406 to Warshaw; U.S. Pat. No. 4,121,326 to Kamiya; U.S. Pat. No. 6,453,521 to Dischler; and U.S. Pat. No. 7,293,334 to Metzger, each of which is hereby incorporated by reference in its entirety. A zipper (or “slide fastener” or “fastener”) includes two sets of interlockable teeth and a slide movable between a first position where a major portion of one of the two sets of teeth is spaced from the other one of the two sets of teeth and a second position where a major portion of the two sets of teeth are interlocked. The zipper 220 (or “slide fastener” or “slide”) is sized, adapted and configured to be movable between a first position for reducing the pressure described above along a varicose vein and to enable a wearer to remove the compression stocking 200A and a second position to enable the body 201A of compression stocking 200A to exert the pressure described above along varicose veins of a wearer of the stocking 200A of the present subject matter.

Features of the present subject matter, described in connection with the knee-high compression sock 100A (FIGS. 10 and 11) and the thigh-high compression stocking 200A (FIGS. 12, 13), including zipper 120 (FIGS. 10, 11) and 220 (FIGS. 12, 13), are applicable to, and hereby incorporated by reference into, each of the knee-high compression sleeve 300 (FIGS. 6, 8) and the thigh-high compression sleeve 400 (FIGS. 5, 7) described above.

A linear pad can be attached to a knee-high compression stocking, e.g., by cutting a stocking on its lateral side from a first predetermined point near the knee to another predetermined point spaced above and near the ankle; and a linear pad can be attached to thigh-high compression stockings by cutting a stocking along its lateral side from a first predetermined point located on a thigh to another predetermined point spaced above and near the ankle. After a spaced-apart pair of linear pads have been attached to a lateral side of a knee-high or a thigh-high compression stocking by sewing the linear pads to a compression stocking longitudinally closely adjacent to the cut in the stocking, an upper portion of each linear pad is sewn to a compression stocking. Upper portions of the linear pads are sewn to the stocking. A zipper can be sewn to a lower portion of a compression stocking. An attached zipper makes a compression stocking easier to put on and remove.

Background for embodiment of the present subject matter shown in FIGS. 10-13: After endovenous ablation procedures are performed on abnormal saphenous veins of a typical patient, significant anatomical and physiological changes occur in legs of a patient. For instance, such patient anatomical and physiological changes often include obliteration of treated superficial veins and of heat-induced thrombus, as well as the relief of venous hypertension, including the eventual disappearance of ankle swelling as well as certain other symptoms. With such positive results with the legs of a patient after a vein ablation procedure, it is believed that compression therapy using compression stockings having linear pads is a better patient option than the traditional graduated compression stockings.

Developing this embodiment of the present subject matter has resulted in my concluding that a compression stocking with linear pads is a better compression therapy compared to graduated compression stockings after an endovenous ablation, because:
1. Delivery of graduated compression stockings high pressure around the ankles is no longer necessary since venous hypertension is relieved after an endovenous ablation.
2. Compression stockings with linear pads can effectively decompress newly treated saphenous veins without the need of firmly compressing the whole leg. This helps a patient's leg recover faster from an endovenous ablation procedure.
3. Compression stockings with linear pads are easier to put on and take off and are more tolerable on sore legs of a patient after a vein ablation procedure.
4. A compression stocking with linear pads is more convenient for a nurse to put on a patient's leg after an endovenous ablation procedure since there is no need for a nurse to use devices, e.g., don and doff, for assistance.

Embodiments of the present subject matter presented and disclosed in connection with FIGS. 10-13 are hereby incorporated into embodiments of the present subject matter disclosed in connection with FIGS. 1-9; and the detailed description disclosed in relation to FIGS. 10-13 is hereby incorporated by reference into disclosure relating to FIGS. 1-9.

Described in detail in this patent specification is compression legwear including elongated pads for controlling symptoms of varicose veins and further including a zipper for enabling a wearer to easily put on and remove the compression legwear. While the present subject matter is described in detail with relation to its present embodiments, the present subject matter is not to be limited to these embodiments. On the contrary, alternatives, changes, and/or modifications shall become apparent to a person of ordinary skill in the art (“POSITA”) after this patent specification and its accompanying figures have been reviewed. Therefore, all such alternatives, changes, and/or modifications are to be considered as constituting a part of the present subject matter insofar as they fall within the spirit and scope of the appended claims as well as claim amendments that may occur.

1. A compression sleeve comprising:
a compression body adapted and configured to be worn by a person having at least one varicose vein, wherein the body includes an upper edge margin and a lower edge margin spaced from the upper edge margin, wherein the body defines a linear opening extending between the upper and lower edge margins;
at least one elongated pad secured to an inner surface portion of the body, wherein the at least one pad overlays a major portion of at least one varicose vein of a person wearing the sleeve, wherein the at least one pad has opposite end portions, wherein one of the end portions is located proximate to one of the upper and lower edge margins and the other end portion is proximate to the other one of the upper and lower edge margins, wherein the at least one pad is adapted and configured to exert a compression of about 20-30 mm Hg to said major portion of said at least one varicose vein of a wearer of the sleeve; and
a slide fastener fixed to the body, overlaying the linear opening defined by the body, and movable between a first position for enabling a person wearing the sleeve to remove the sleeve and a second position spaced from the first position for causing compression to exert a compression of about 20-30 mm Hg to said major portion of said at least one varicose vein of a wearer of the sleeve.

2. The sleeve of claim 1, wherein the compression body is dimensioned, adapted, and configured to contact a portion of skin of a person wearing the sleeve and to exert a compression of about 15-20 mm Hg to said skin portion of a person wearing the sleeve.

3. The sleeve of claim 1, wherein the slide fastener is sewn or stitched to the body, wherein the at least one pad is adhesively fixed to the inner surface portion of the body.

4. The sleeve of claim 1, wherein the sleeve has a length ranging from about 10 centimeters to about 150 centimeters.

5. The sleeve of claim 1, wherein the sleeve has a length ranging from about 20 centimeters to about 140 centimeters.

6. The sleeve of claim 1, wherein the sleeve has a length ranging from about 10 centimeters to about 80 centimeters.

7. The sleeve of claim 1, wherein the body is made of spandex blended with a material selected from the group consisting of cotton, polyester, nylon, and combinations thereof.

8. The sleeve of claim 1, including at least one other elongated pad fixed to another inner surface portion of the body, wherein the at least one other pad overlays a major portion of at least one other varicose vein of a person wearing the sleeve, wherein the at least one other pad has opposite end portions, wherein one of the end portions of the at least one other pad is located proximate to one of the upper and lower edge margins, wherein the other end portion of the at least one other pad is located proximate to the other one of the upper and lower edge margins, and wherein the at least one other pad is dimensioned, adapted and configured to exert a compression of about 20-30 mm Hg to said major portion of said at least one other varicose vein of a wearer of the sleeve.

9. A compression sock comprising:
a compression body adapted and configured to be worn by a person having at least one varicose vein, wherein the body defines an upper portion located proximate to a knee of a wearer of the sock, wherein the body defines a lower portion spaced from the upper portion, wherein the lower portion is sized and configured to fit a foot of a wearer of the sock, wherein the body defines a linear opening extending between the knee and the foot of a wearer of the sock;
at least one elongated pad secured to an inner surface portion of the body, wherein the at least one pad overlays a major portion of at least one varicose vein of a person wearing the sock, wherein the at least one pad has opposite end portions, wherein one of the end portions is located proximate to one of the knee and the foot of a wearer of the sock and the other one of the end portions is proximate to the other one of the knee and the foot of a wearer of the sock, wherein the at least one pad is dimensioned, adapted and configured to exert a compression of about 20-30 mm Hg to said major portion of said at least one varicose vein of a wearer of the sock; and
a slide fastener, secured to the body, overlaying the linear opening defined by the body, and movable between a first position for enabling a person wearing the sock to remove the sock and a second position spaced from the first position for causing the at least one pad to exert a compression of about 20-30 mm Hg to said major portion of said at least one varicose vein of a wearer of the sock.

10. The sock of claim 9, wherein the slide fastener is a zipper.

11. The sock of claim 9, wherein the compression body is dimensioned, adapted, and configured to contact a portion of skin of a person wearing the sock and to exert a compression of about 15-20 mm Hg to said skin portion of a person wearing the sock.

12. The sock of claim 9, wherein the slide fastener is sewn or stitched to the body, wherein the at least one pad is adhesively fixed to the inner surface portion of the body.

13. The sock of claim 9, wherein the body is made of spandex blended with a material selected from the group consisting of cotton, polyester, nylon, and combinations thereof.

14. The sock of claim 9, further including at least one other elongated pad secured to another inner surface portion of the compression body, wherein the at least one other pad overlays a major portion of at least one other varicose vein of a person wearing the sock, wherein the at least one other elongated pad has opposite end portions, wherein one of the end portions of the at least one other elongated pad is located proximate to one of the knee and the foot of a wearer of the sock, wherein the other end portion of the at least one other elongated pad is located proximate to the other one of the knee and the foot of a wearer of the sock, wherein the at least one other elongated pad is dimensioned, adapted, and configured to exert a compression of about 20-30 mm Hg to said major portion of said at least one other varicose vein of a person wearing the sock.

15. A compression stocking comprising:
a compression body adapted and configured to be worn by a person having at least one varicose vein, wherein the body defines an upper portion sized, adapted, and configured to fit a thigh of a wearer of the stocking, wherein the compression body also defines a lower portion spaced from the upper portion, wherein the lower portion is sized, adapted, and configured to fit a foot of a wearer of the stocking, wherein the compression body further defines a linear opening extending between the thigh and the foot of a wearer of the stocking;
at least one elongated pad secured to an inner surface portion of the body, wherein the at least one elongated pad overlays a major portion of at least one varicose vein of a person wearing the stocking, wherein the at least one elongated pad has opposite end portions, wherein one of the end portions of the at least one elongated pad is located proximate to one of the thigh and the foot of a person wearing the stocking and the other one of the end portions of the at least one elongated pad is located proximate to the other one of the thigh and the foot of a person wearing the stocking, wherein the at least one pad is dimensioned, adapted, and configured to exert a compression of about 20-30 mm Hg to said major portion of said at least one varicose vein of a wearer of the stocking; and
a slide fastener, fixed to the body, overlaying the linear opening, and movable between a first position for enabling a person wearing the stocking to remove the stocking and a second position spaced from the first position for causing the at least one elongated pad to exert a compression of about 20-30 mm Hg to the major portion of said at least one varicose vein of a person wearing the stocking.

16. The stocking of claim 15, wherein the slide fastener is a zipper.

17. The stocking of claim 15, wherein the compression body is dimensioned, adapted, and configured to contact a portion of skin of a person wearing the stocking and exert a compression of about 15-20 mm Hg to said skin portion of a person wearing the stocking.

18. The stocking of claim 16, wherein the zipper is sewn or stitched to the body, and wherein the at least one pad is adhesively fixed to the inner surface portion of the body.

19. The stocking of claim 15, wherein the body is spandex blended with a material selected from the group consisting of cotton, polyester, nylon, and combinations thereof.

20. The stocking of claim 15, including at least one other elongated pad secured to another inner surface portion of the body, wherein the at least one other elongated pad overlays a major portion of at least one other varicose vein of a person wearing the sock, wherein the at least one other elongated pad has opposite end portions, wherein one of the end portions of the at least one other elongated pad is located proximate to one of the thigh and the foot of a wearer of the sock, wherein the other end portion of the at least one other elongated pad is located proximate to the other one of the thigh and the foot of a wearer of the sock, wherein the at least one other elongated pad is dimensioned, adapted, and configured to exert a compression of about 20-30 mm Hg to said major portion of said at least one other varicose vein of a person wearing the stocking.
